(19) United States
(12) Patent Application Publication
(10) Pub. No.: US 2006/0030788 A1
(43) Pub. Date: Feb. 9, 2006

(54) Apparatus and method for extracting bodily fluid utilizing a flat lancet

(76) Inventors: Daniel Wong, Sunnyvale, CA (US); Charles C. Raney, Camdenton, MO (US); Paul Patel, Sunnyvale, CA (US); Steven N. Roe, San Mateo, CA (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 10/911,252

(22) Filed: Aug. 4, 2004

(51) Int. Cl. A61B 5/00

(52) U.S. Cl. 600/583

(57) ABSTRACT

A bodily fluid sampling device is operable to rupture the skin surface at normal and oblique angles utilizing a flat lancet of specified thickness and at least one cutting surface inclined less than eighty degrees (80°) from the longitudinal axis of the flat lancet. The flat lancet thickness and cutting surface geometry minimize pain while maximizing the amount of emerged bodily fluid. The device limits the depth to which the flat lancet penetrates the skin to further minimize pain and may urge the bodily fluid from the rupture site. The bodily fluid emerging from the rupture site is transported to a test device that tests the bodily fluid for particular properties and characteristics.

### BACKGROUND

The present invention generally relates to bodily fluid sampling devices and, more specifically, but not exclusively, concerns bodily fluid sampling devices with lancets of particular geometry configured to penetrate the skin at a particular angle to reduce pain and improve fluid flow from the body.

The acquisition and testing of bodily fluids is useful for many purposes, and continues to grow in importance for use in medical diagnosis and treatment, and in other diverse applications. In the medical field, it is desirable for lay operators to perform tests routinely, quickly and reproducibly outside of a laboratory setting. Testing can be performed on various bodily fluids, and for certain applications is particularly related to the testing of blood and/or interstitial fluid. Such fluids can be tested for a variety of characteristics of the fluid, or analytes contained in the fluid, in order to identify a medical condition, determine therapeutic responses, assess the progress of treatment, and the like.

The testing of bodily fluids basically involves the steps of obtaining the fluid sample, transferring the sample to a test device, conducting a test on the fluid sample, and displaying the results. Advantages are realized when some or all of the steps involved in testing bodily fluid are automated. Automated test steps can produce results that are more consistent than steps that are not automated.

A common technique for collecting a bodily fluid sample is to breach the skin surface to bring the fluid to the skin surface. Round instruments, such as needles, may be used to breach the skin surface; however, round instruments typically result in the collection of relatively small amounts of bodily fluid and can be relatively expensive to produce. A lancet, knife or other cutting instrument may also be used to form an incision in the skin. Flat lancets can result in the collection of larger amounts of bodily fluid than round instruments, but may cause more pain than round instruments. However, the level of pain can be minimized by use of particular lancet tip geometries. Additionally, automating the skin breaching can more consistently create appropriately sized and shaped incisions for testing, decrease the need for additional lancing, and result in less pain overall.

Regardless of the instrument, the resulting bodily fluid sample is then collected. The fingertip is frequently used as the fluid source, although alternate sampling sites, such as the palm of the hand, forearm, earlobe and the like, may be useful to decrease the pain associated with breaching the skin surface. However, alternate sampling sites tend to produce lesser amounts of blood.

Although some amount of bodily fluid naturally emerges from a rupture in the skin surface, additional fluid is frequently urged, or expressed, from the rupture site to increase the total amount of emerged bodily fluid. An example method of expressing includes applying pressure to the area surrounding the rupture to milk or pump the fluid from the rupture site. Mechanical devices may also be used to express bodily fluid from the rupture.

The acquisition of the produced bodily fluid, or “sampling” of the fluid, can take various forms. For example, once the fluid specimen emerges from the breach site, a sampling device is placed into contact with the fluid. Example sampling devices are capillary tubes, vacuum tubes, and pumps, which may be used individually or in various combinations. Certain advantages can be realized using flat lancets as flat lancets can be readily assimilated into integrated sampling devices.

The bodily fluid sample may be analyzed for a variety of properties or components. For example, such analysis may be directed to hematocrit, blood glucose, coagulation, lead, iron, etc. Testing systems include such means as optical (e.g., reflectance, absorption, fluorescence, Raman, etc.), electrochemical, and magnetic means for analyzing the sampled fluid. Typically, a test system takes advantage of a reaction between the bodily fluid to be tested and a reagent present in the test system.

Problems remain and a need exists in the bodily fluid sampling device field for an improved sampling device utilizing a relatively inexpensive incision forming member with appropriate size, shape and penetration geometry to minimize pain while maximizing the amount of emerged bodily fluid. A further need exits for an incision forming member that is readily assimilated into an integrated sampling device. The present invention addresses these and other difficulties in the bodily fluid sampling device field.

### SUMMARY

One form of the present invention concerns a bodily fluid sampling device that includes a flat incision forming member adapted to form an incision in skin. The incision forming member includes a specified thickness and tip design and penetrates the skin at an angle to a pre-determined depth. A retraction mechanism is coupled to the incision forming member to retract the incision forming member from the skin.

Utilizing incision forming members that are relatively thin, such as when thickness is less than 0.05 mm, results in a tendency for the members to bend or break. Utilizing incision forming members that are relatively thick generally results in increased levels of pain as the member penetrates the skin. Utilizing incision forming members with a cutting edge parallel to the skin surface can further result in increased pain due to tearing of the skin as the member penetrates the skin; while utilizing incision forming members with a cutting edge nearing ninety degrees (90°) from the skin surface results in members that are prone to bending or breaking and members that must penetrate further to create a reasonable amount of emerged bodily fluid. If blood is the desired bodily fluid, utilizing a small incision forming member that penetrates the skin orthogonally can result in no blood being released. Furthermore, utilizing incision forming members that are not at least partially automated can result in additional pain for the test subject.

In one embodiment of the present invention, a flat lancet is adapted to form a breach in the skin surface. The lancet includes a longitudinal axis, a thickness, a width, a first cutting edge adapted to penetrate the skin, and a second cutting edge also adapted to penetrate the skin. The thickness is at least 0.05 millimeters and at most 0.15 millimeters, and the width is at least 0.1 millimeters and at most 1.5 millimeters to minimize the pain associated with the lancet penetrating the skin. The first and second cutting edges are each inclined at least thirty degrees (30°) and at most forty degrees (40°) from the longitudinal axis and the thickness. An actuation member is attached to the flat lancet which moves the first cutting edge to penetrate the skin.

In another embodiment of the present invention, a flat lancet is adapted to form a breach in the skin surface. The lancet includes a longitudinal axis, a thickness, and a first cutting edge which is adapted to penetrate the skin. The first cutting edge is inclined less than eighty degrees (80°) from the longitudinal axis and the thickness is at least 0.05 millimeters and at most 0.3 millimeters to minimize the pain associated with the lancet penetrating the skin. Additionally, an actuation member is attached to the flat lancet. The actuation member moves the first cutting edge to penetrate the skin.

In yet another embodiment of the present invention, a method is used, whereby a sampling device that includes a generally planar lancet is placed in contact with the skin. The generally planar lancet has a thickness of at least 0.05 millimeters and at most 0.3 millimeters, and has at least one cutting edge. The method also has the generally planar lancet moved in relation to the skin surface and along a translation axis. The method further has the generally planar lancet penetrate the skin surface, where at least one cutting edge of the generally planar lancet is inclined less than eighty degrees (80°) from the translation axis.

In still another embodiment of the present invention, an incision forming member is adapted for cutting the skin surface. The incision forming member includes a side surface and at least one cutting facet inclined less than eighty degrees (80°) from the side surface, and the incision forming member has a thickness greater than 0.05 millimeters and less than 0.3 millimeters. Furthermore, a means for moving at least a portion of the incision forming member below the skin surface at an incision location is utilized.

In a still further embodiment of the present invention, a flat lancet for rupturing the skin at a rupture site is used. The flat lancet comprises a first side and a cutting surface. The cutting surface is produced by photoetching, where the photoetching is performed on the first side of the flat lancet. Additionally, an actuation member is used to move the flat lancet to rupture the skin at the rupture site.

Further forms, objects, features, aspects, benefits, advantages and embodiments of the present invention will become apparent from a detailed description of the drawings provided herewith.

### BRIEF DESCRIPTION OF THE DRAWINGS

FIG. 1 is a front view of a bodily fluid sampling device according to one embodiment of the present invention with the lancet in an extended position.

FIG. 2 is a cross-sectional front view of the device of FIG. 1 positioned orthogonally to a skin surface.

FIG. 3 is a front view of a lancet according to one embodiment of the present invention.

FIG. 4 is a side view of the lancet depicted in FIG. 3.

FIG. 5 is a front view of a lancet according to another embodiment of the present invention.

FIG. 6 is a side view of the lancet depicted in FIG. 5.

FIG. 7 is a front view of a lancet according to an additional embodiment of the present invention.

FIG. 8 is a side view of the lancet depicted in FIG. 7.

FIG. 9 is a front view of a lancet according to a further embodiment of the present invention.

FIG. 10 is a side view of the lancet depicted in FIG. 9.

FIG. 11 is a front view of a bodily fluid sampling device according to yet another embodiment of the present invention.

FIG. 12 is a cross-sectional front view of the device of FIG. 11 positioned obliquely to the skin surface FIG. 13 is a cross-sectional side view of the lancet depicted in FIG. 3 orthogonally penetrating the skin surface.

FIG. 14 is a cross-sectional front view of the lancet of FIG. 5 obliquely penetrating the skin surface.

FIG. 15 is a front view of a bodily fluid sampling device according to yet a further embodiment of the present invention.

FIG. 16 is a cross-sectional side view of the device of FIG. 15 as viewed along line 16-16.

FIG. 17 is a cross-sectional side view of the device of FIG. 15 as viewed along line 17-17.

FIG. 18 is a cross-sectional side view of the device of FIG. 15 as viewed along line 18-18.

FIG. 19 is a front view of a lancet according to another embodiment of the present invention.

FIG. 20 is a front view of a lancet according to an additional embodiment of the present invention.

FIG. 21 is a front view of a lancet according to a further embodiment of the present invention.

FIG. 22 is a front view of a lancet according to yet another embodiment of the present invention.

FIG. 23 is a cut-away rear view of the lancet depicted in FIG. 22.

FIG. 24 is a cut-away side view of the lancet depicted in FIG. 22.

FIG. 25 is a front view of a lancet according to yet a further embodiment of the present invention.

FIG. 26 is a cut-away side view of the lancet depicted in FIG. 20 according to one embodiment of the present invention.

FIG. 27 is a cut-away side view of the lancet depicted in FIG. 20 according to another embodiment of the present invention.

FIG. 28 is a cut-away side view of the lancet depicted in FIG. 20 according to a further embodiment of the present invention.

FIG. 29 is a cut-away side view of the lancet depicted in FIG. 20 according to yet another embodiment of the present invention.

FIG. 30 is a cut-away side view of the lancet depicted in FIG. 20 according to still a further embodiment of the present invention.

FIG. 31 is a graphic representation of example test data depicting interrelation between lancet tip angle, pain and penetration depth setting.

### DESCRIPTION OF THE SELECTED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. Certain embodiments of the invention are shown in detail, although it will be apparent to those skilled in the relevant art that some features that are not relevant to the present invention may not be shown for the sake of clarity.

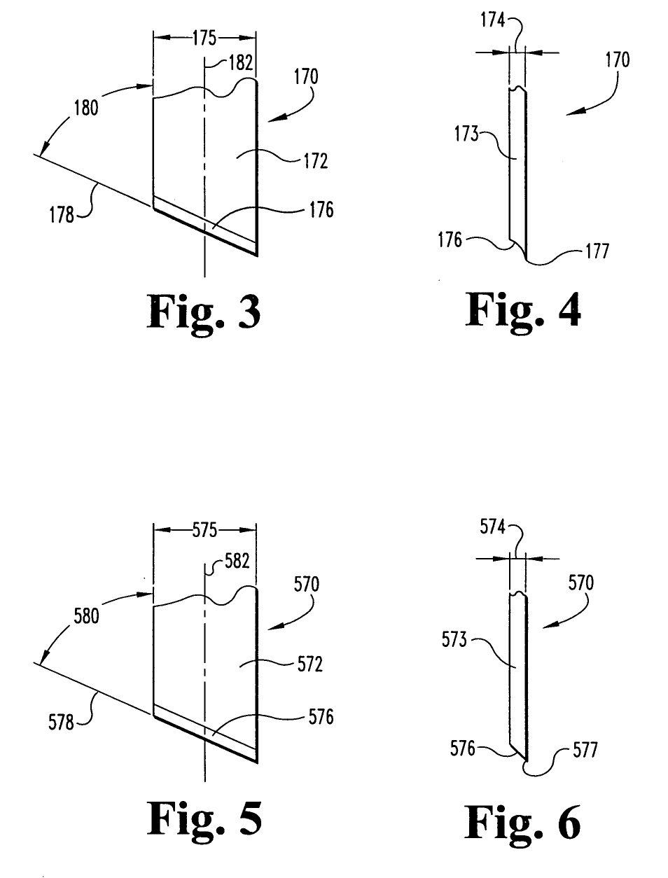

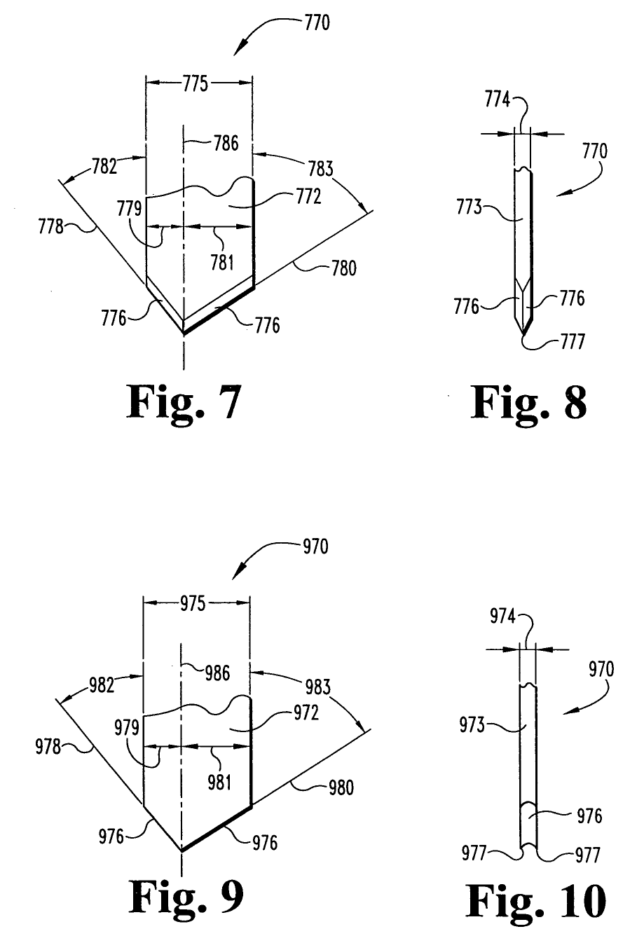

While developing improved lancets to maximize the amount of expressed fluid while minimizing pain, various lancet parameters were tested, including lancet tip geometry and tissue penetration angle. Although certain results were anticipated, such as an increasing level of pain as the overall size of the lancet penetrating portion increased, other results were unanticipated, such as certain flat lancet tip geometries producing less pain than round lancets as penetration depth setting increased. Other unexpected results include a lower than expected limit to the maximum tip angle and the discovery that angling the penetration angle can increase the amount of emerged bodily fluid without appreciably increasing pain. It was also discovered that symmetrical double cutting edge flat lancets (for example, FIGS. 7-10 and 19-25 with equal tip angles) having thicknesses of at least 0.05 millimeters and at most 0.15 millimeters, widths of at least 0.1 millimeters and at most 1.5 millimeters, and both cutting edges symmetrically inclined at least fifteen degrees (15°) and at most twenty degrees (20°) tended to maximize the amount of emerged bodily fluid while minimizing the level of pain for certain embodiments. It was further discovered that single cutting edge flat lancets (for example, FIGS. 3-6) having thicknesses of at least 0.05 millimeters and at most 0.15 millimeters, widths of at least 0.1 millimeters and at most 1.5 millimeters, and with the cutting edge inclined at least thirty degrees (30°) and as most forty degrees (40°) tended to maximize the amount of emerged bodily fluid while minimizing the level of pain for certain embodiments.

Bodily fluid sampling devices according to the present invention are operable to form an incision with a generally flat lancet at both fingertip and alternate sites. The flat lancet includes at least one angled cutting surface, or cutting facet, and a particular lancet thickness. The devices can be configured such that the lancet penetrates the skin orthogonally or at an oblique angle. In one particular embodiment, the device includes a skin contacting portion configured to result in the lancet penetrating the skin at an oblique angle when the skin contacting portion is placed flat against the skin surface. Upon actuation, the flat lancet moves toward and penetrates the skin. The flat lancet can automatically stop at a predetermined depth and can be later extracted from the skin. The use of the flat lancet with a particular tip design and thickness in conjunction with angled penetration to a predetermined depth minimizes the pain associated with lancing while maximizing the amount of bodily fluid released due to the lancing.

Certain embodiments of the current invention include an expressing portion that urges bodily fluids from the lancing site, an integrated testing portion that tests the sample of bodily fluid for selected properties, a sampling portion that moves the bodily fluid from the lancing site to the testing portion, and a display to show the results to the user. These features are described in the following references, which are incorporated herein by reference in their entirety: U.S. application Ser. No. 08/857,680, filed May 16, 1997, now U.S. Pat. No. 5,879,311; U.S. application Ser. No. 08/858,042, filed May 16, 1997, now U.S. Pat. No. 5,951,492; U.S. application Ser. No. 08/858,043, filed May 16, 1997, now U.S. Pat. No. 5,951,493; U.S. application Ser. No. 09/238,158, filed Jan. 28, 1999, now U.S. Pat. No. 6,183,489; U.S. application Ser. No. 09/963,967, filed Sep. 26, 2001, now U.S. Pat. No. 6,616,616; and U.S. application Ser. No. 10/254,314, filed Sep. 25, 2002, published as U.S. Patent Application Publication No. 2004/0059256 A1.

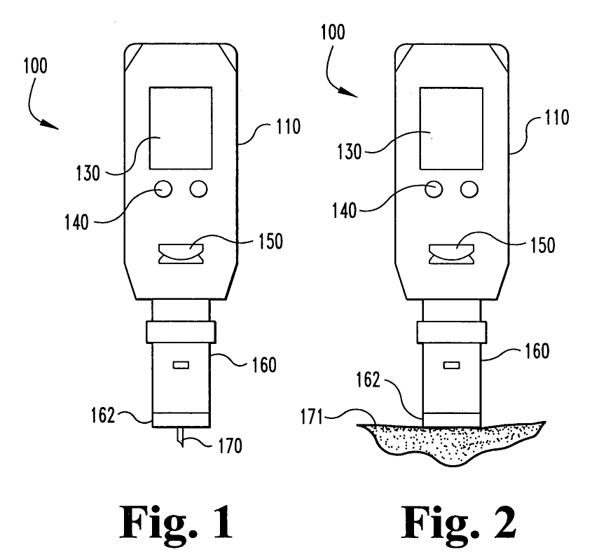

A bodily fluid sampling device 100 according to one embodiment, among others, of the present invention is illustrated in FIGS. 1 and 2. The sampling device 100 includes a testing portion 110 for testing samples of bodily fluid. The testing portion 110 further includes a display 130 for displaying test results, an actuation trigger 140 for initiating sampling, and a calibration interface 150 for calibrating the sampling device 100.

The sampling device 100 further includes a lancing portion 160. The lancing portion 160 includes a skin contacting portion 162 for positioning the sampling device on the skin, and a flat lancet 170 for penetrating the skin. In one embodiment, actuating the actuation trigger 140 results in an actuation member moving lancet 170 to penetrate the skin. Although FIGS. 1 and 2 show the testing portion 110 as being integrated with the lancing portion 160, it is contemplated that in other embodiments, the lancing portion 160 and testing portion 110 can be separate components.

It should be appreciated that the sampling device 100 may utilize numerous lancets. Sampling device 100 may further utilize a cassette design where at least one lancet is housed in each cassette and the cassettes are moved into a lancing position prior to the lancets being moved to rupture the skin. The cassette design may further be used in conjunction with at least one of a sampling device, an expression device and a testing portion.

In use, the sampling device 100 is positioned on the body such that the skin contacting portion 162 is flush against a skin surface 171 (FIG. 2). Once properly positioned, the actuation trigger 140 is activated and the flat lancet 170 is forcibly moved to penetrate the skin surface 171. This movement may occur along a translation axis that is straight or curved. Portions of the lancet may be held motionless with the lancet tip pivoting toward the skin surface. As should be understood, the lancet can be driven toward the skin surface through a number of mechanisms, such as, for example, springs, hydraulics, pneumatics, chemical reactions, electric motor, or a hammer striking the lancet. The actuation trigger 140 is activated by the user depressing a button. Other embodiments activate the actuation trigger 140 by other mechanisms, such as by activating when the skin contacting portion 162 is pressed against the skin.

In the depicted example, the flat lancet 170 penetrates the skin surface 171 orthogonally relative to the skin surface 171. The flat lancet 170 is configured to penetrate the skin surface 171 to a predetermined penetration depth. By way of nonlimiting example, the penetration depth may range from 0.5 to 2.0 mm. Various methods may be utilized to stop the flat lancet 170 at the predetermined penetration depth, such as an adjustable mechanical stop. As a result of the incision, bodily fluid emerges from the skin. Example bodily fluids include blood and interstitial fluid; however, it should be appreciated that other types of bodily fluid may be sampled. The bodily fluid can further be expressed to increase the total amount of emerged bodily fluid.

A portion, or all, of the emerged bodily fluid is sampled, or transported, to the testing portion 110 where it is tested for particular properties. Various methods may be used to transport the bodily fluid to the testing portion, such as vacuum suction and capillary action. The bodily fluid may be transported from the skin surface, below the skin surface or directly from a blood vessel to the testing portion 110. The lancet 170 may be retracted from the skin surface prior to, during, or after sampling the bodily fluid. Alternatively, the lancet 170 may be moved while in the skin to facilitate expression of bodily fluid.

The results of the bodily fluid testing are displayed on the display 130. The calibration interface 150 may be used to calibrate the testing device based on the particular batch of test strips being used. For example, a computer chip containing calibration information may be included with each set of test strips and received into calibration interface 150 prior to use.

The sampling device 100 may be comprised of a variety of materials, such as, metal, plastic, or composites. Typically, the sampling device 100 is sized to fit into and be operated by an average person's hand. The flat lancet 170 is typically comprised of material that maintains its sharpness as it penetrates the skin, such as surgical grade stainless steel; however, other materials may be utilized such as plastics or ceramics.

Referring to FIGS. 3 and 4, an example embodiment of the flat lancet 170 is depicted. The flat lancet 170 has two opposing, generally parallel surfaces 172 with two opposing side surfaces 173. The two opposing, generally parallel surfaces 172 and the two opposing side surfaces 173 are generally planar, although they may be curved or angled. The mean distance between generally parallel surfaces 172 is the lancet thickness 175, and the mean distance between side surfaces 173 is the lancet width 175. The lancet thickness 174 is generally less than the lancet width 175.

The flat lancet 170 is bounded on yet another side by the cutting surface 176. The intersection of the cutting surface 176 and one of the generally parallel surfaces 172 forms a cutting edge 177. The cutting edge 177 defines a cutting edge axis 178. The cutting edge axis 178 is offset by a tip angle 180 from the longitudinal axis 182 of the lancet 170. The longitudinal axis 182 generally defines the long portion of the lancet 170; however, other example embodiments have the longitudinal axis 182 parallel, or tangential, to the thrust axis, where the thrust axis defines the movement of the lancet 170 as it penetrates the skin surface 171. FIG. 3 depicts the tip angle 180 as being measured from the side of the lancet 170, where the side from which the tip angle 180 is being measured is parallel to the longitudinal axis 182 of the lancet 170. It is understood that other embodiments of the present invention have, by way of nonlimiting example, the sides angled with respect to the longitudinal axis 182.

The cutting surface 176 may be formed by various methods, such as photoetching, mechanical grinding, mechanical stamping, or laser shaping, by way of nonlimiting examples. The cutting surface 176 is curved, although other example embodiments have cutting surfaces that are planar or angled. Different methods of forming the cutting surface 176 may be utilized with different lancet thicknesses. It was discovered that photoetching tends to be suited for relatively thin lancets, approximately 0.05 to 0.1 mm thick, while mechanical grinding or stamping tends to be suited for thicker lancets, approximately 0.1 to 0.2 mm thick.

Photoetching, a type of lithography, generally includes the placement of a patterned layer of photoresist on one side of lancet material and subsequent etching of the same side. The etching may be achieved by application of an etchant, or etching solution, to the same side of the lancet material to which the photoresist was placed. The etchant dissolves the lancet material but does not dissolve the photoresist, thereby removing the portions of lancet material not covered by the photoresist. The photoresist is then stripped from the etched lancet material leaving only the etched lancet material. This discussion of photoetching is illustrative and is not limiting. It is understood that other methods of lithography and etching may be used to produce lancet 170.

Photoetching can result in straight, curved or angled tapering or sloping of the lancet material from one generally parallel surface 172 to the other, and can result in one or a combination of curved, planar, angled, concave or convex shapes for cutting surface 176. It should be appreciated that photoetching may be performed on more than one side of the flat lancet. Single sided photoetching occurs when the photoresist and etchant are applied to one side of the lancet material. Double sided photoetching occurs when the photoresist and etchant are applied to two sides of the lancet material, and can result in tapering from both sides of lancet 170. It was discovered that single sided photoetching tends to be suited for producing cutting surface 176, although double sided photoetching may also be utilized.

As mentioned above, alternate sampling sites, such as the palm of the hand, forearm, earlobe and the like, may be useful for sampling because lancing these sites may be less painful than lancing at traditional fingertip sites. However, a drawback with lancing at alternate sampling sites is that the amount of fluid that can emerge from an incision formed in an alternate sampling site is relatively small when compared to fingertip sites. The flat lancet 170 is useful in lancing at alternate sampling sites, as well as at fingertip sites, as it minimizes the pain associated with lancing while maximizing the total amount of emerged bodily fluid.

For the set lancet width 172, it was discovered that the pain associated with a lancet penetrating the skin may be minimized by varying the tip angle 180 and the lancet thickness 174. Larger lancet thickness and lancet width increase the probability of pain as the lancet penetrates the skin since the geometrically larger lancet has a higher probability of affecting or severing at least one nerve.

In general, single cutting edge lancets with smaller tip angles penetrate the skin tissue more easily with less tearing and produce less pain. However, it was discovered that if the tip angle became too small, the penetration depth required to produce the desired amount of emerged bodily fluid increased to a point where the pain levels became unacceptable. Furthermore, it was realized that when the tip angles became too small, the tendency of the lancet to bend during use increased, thereby increasing pain levels.

Conversely, single cutting edge lancets with larger tip angles tend to require more force to penetrate the skin than lancets with smaller tip angles resulting in additional pressure sensing nerves being affected and increasing the overall sensation of pain. It was expected that the pain levels associated with penetration of lancet 170 would become unacceptable for tip angles 180 nearing ninety degrees (90°). However, it was discovered that, not only were there unacceptably high levels of pain, but that there was also very poor penetration of lancet 170 at tip angles 180 as unexpectedly low as sixty degrees (60°). Testing further revealed that satisfactory levels of pain and emerged bodily fluid were realized with tip angle 180 at least thirty degrees (30°) and at most forty degrees (40°) for certain example embodiments.

It was discovered that the lancet width 175 can vary from 0.1 to 1.5 mm. It was further discovered that the lancet thickness 174 can vary from 0.05 mm to 0.3 mm, and in particular embodiments from 0.05 to 0.15 mm. In certain embodiments, a lancet thickness 174 between 0.05 and 0.1 mm may produce favorable results when flat lancet 170 is formed utilizing photoetching. In other embodiments, a lancet thickness 174 between 0.1 and 0.2 mm may produce favorable results when flat lancet 170 is formed utilizing mechanical stamping.

The tip angle 180 is at most eighty degrees (80°). It was discovered that the tip angle can particularly range from ten to sixty degrees (10° to 60°), and more particularly from thirty to forty degrees (30° to 40°) to minimize pain associated with skin penetration while maximizing the total amount of emerged bodily fluid. It was additionally discovered that very high levels of pain and poor tip penetration into the skin resulted when the tip angle 180 was at least sixty degrees (60°).

Table 1 is a representation of example test data depicting the interrelation between lancet tip angle, blood volume, penetration depth and pain. As can be seen from the example data for a 0.076 mm thick flat lancet, it was discovered that the frequency of the test subject experiencing a pain rating of 2 (“discomfort”) or greater increased from 8% to 16% as lancet tip angle increased from thirty degrees (30°) to fifty degrees (50°). The example data further reflects that blood volume decreased from 10.94 μl to 6.89 μl as the penetration depth decreased from 0.84 mm to 0.68 mm.

TABLE 1

| | | Depth of | % Pain |
|---|---|---|---|
| Lancet Tip | Blood | Lancet | Rating at |
| Angle | Volume | Penetration | 2 or greater |
| (degrees) | (μL) | (mm) | (occurrences) |
| 30 | 10.94 | 0.84 | 8 |
| 40 | 10.05 | 0.82 | 13 |
| 50 | 6.89 | 0.68 | 16 |

| | |
|---|---|
| Lancet Type: | Flat Lancets |
| Lancet Thickness: | 0.076 mm |
| Lancet Manufacture: | Photo-etching |
| Depth Setting: | 1.0, if unsuccessful then 1.5 |
| Test Site: | Fingertip |
| Subjects/N: | Diabetics & Non-Diabetics/N = 40 |
| Pain Rating Scale: | 0 = No pain/1 = Sensation/ |
| | 2 = Discomfort/3 = Moderate pain/ |
| | 4 = Severe pain |

Referring to FIGS. 5 and 6, another embodiment of a flat lancet 570 that can be used in the sampling device 100 is depicted. The flat lancet 570 has two opposing, generally parallel surfaces 572 with two opposing side surfaces 573. The two opposing generally parallel surfaces 572 and the two opposing side surfaces 573 are generally planar, although they may be curved or angled. The mean distance between generally parallel surfaces 572 is the lancet thickness 574, and the mean distance between side surfaces 573 is the lancet width 575. Generally, the flat lancet 570 has the lancet thickness 574 less than the lancet width 575.

The flat lancet 570 is bounded on yet another side by the cutting surface 576. The intersection of the cutting surface 576 and one of the generally parallel surfaces 572 forms a cutting edge 577. The cutting edge 577 defines a cutting edge axis 578. The cutting edge axis 578 is offset by a tip angle 580 from the longitudinal axis 582 of the lancet 570. The longitudinal axis 582 generally defines the long portion of the lancet 570. FIG. 5 depicts the tip angle 580 as being measured from the side of the lancet 570, where the side from which the tip angle 580 is being measured is parallel to the longitudinal axis 582 of the lancet 570. It is understood that other embodiments of the present invention have, by way of nonlimiting example, the sides angled with respect to the longitudinal axis 582.

The cutting surface 576 may be formed by various methods, such as photoetching, mechanical grinding, mechanical stamping, or laser shaping, by way of nonlimiting examples. The cutting surface 576 is planar, although other example embodiments have cutting surfaces that are curved or angled.

The flat lancet 570 minimizes the pain associated with lancing while maximizing the total amount of emerged bodily fluid. It was discovered that the lancet width 575 can vary from 0.1 to 1.5 mm. For the set lancet width 575, the pain associated with a lancet penetrating the skin may be minimized by varying the tip angle 580 and the lancet thickness 574.

The tip angle 580 is at most eighty degrees (80°). It was also discovered that the tip angle can range particularly from ten to sixty degrees (10° to 60°), and more particularly from thirty to forty degrees (30° to 40°) to minimize pain associated with skin penetration while maximizing the total amount of emerged bodily fluid. It was additionally discovered that very high levels of pain and poor tip penetration into the skin resulted when the tip angle 580 was at least sixty degrees (60°).

It was further discovered that the lancet thickness 574 can vary from 0.05 mm to 0.3 mm, and in particular embodiments from 0.05 to 0.15 mm. In certain embodiments, a lancet thickness 574 between 0.05 and 0.1 mm may produce favorable results when flat lancet 570 is formed utilizing photoetching. In other embodiments, a lancet thickness 574 between 0.1 and 0.2 mm may produce favorable results when flat lancet 570 is formed utilizing mechanical stamping.

As shown in FIGS. 7 and 8, a further example embodiment of a flat lancet 770 is depicted. The flat lancet 770 has two opposing, generally parallel surfaces 772 with two opposing side surfaces 773. The two opposing generally parallel surfaces 772 and the two opposing side surfaces 773 are generally planar, although they may be curved or angled. The mean distance between generally parallel surfaces 772 is the lancet thickness 774, and the mean distance between side surfaces 773 is the lancet width 775. Generally, the flat lancet 770 has the lancet thickness 774 less than the lancet width 775.

The flat lancet 770 is bounded on yet another side by the cutting surfaces 776. The intersection of the cutting surfaces 776 forms the cutting edges 777. There are four cutting surfaces 776 and two cutting edges 777. One cutting edge 777 defines a first cutting edge axis 778 and a first cutting edge width 779, and the remaining cutting edge 777 defines a second cutting edge axis 780 and a second cutting edge width 781. The first cutting edge axis 778 is offset by a first tip angle 782 from the longitudinal axis 786 of the lancet 770, and the second cutting edge axis 780 is offset by a second tip angle 783 from the longitudinal axis 786 of the lancet 770.

The longitudinal axis 786 generally defines the long portion of the lancet 770; however, other example embodiments have the longitudinal axis 786 parallel, or tangential, to the thrust axis, where the thrust axis defines the movement of the lancet 770 as it penetrates the skin surface 171.

FIG. 7 depicts the tip angles 782 and 783 as being measured from the sides of the lancet 770, where the sides from which the tip angles 782 and 783 are being measured is parallel to the longitudinal axis 786 of the lancet 770. It is understood that other embodiments of the present invention have, by way of nonlimiting example, the sides angled with respect to the longitudinal axis 786.

The tip of lancet 770 may be symmetric or asymmetric, although in some embodiments a symmetric tip may be preferable for maximizing blood flow while minimizing pain. An asymmetric tip may include, but not be limited to, first cutting edge width 779 and the second cutting edge width 781 being different, cutting surfaces 776 having unequal surface areas, or tip angle 782 and tip angle 783 being different.

The four cutting surfaces 776 may be formed by various methods, such as photoetching, mechanical grinding, mechanical stamping, or laser shaping, by way of nonlimiting examples. The cutting surfaces 776 are generally planar, although other example embodiments have cutting surfaces that are curved or angled. It was discovered that double sided photoetching tends to be suited for producing cutting surface 776, although single sided photoetching may also be utilized.

The flat lancet 770 minimizes the pain associated with lancing while maximizing the amount of emerged bodily fluid. It was discovered that lancet width 775 can vary from 0.1 to 1.5 mm. For the set lancet width 775, the pain associated with a lancet penetrating the skin may be minimized by varying the tip angles 782 and 783, as well as the lancet thickness 774. The tip angles 782 and 783 are less than eighty degrees (80°). It was also discovered that the tip angles 782 and 783 can individually range particularly from ten to sixty degrees (10° to 60°), and more particularly from fifteen to twenty degrees (15° to 20°) to minimize pain associated with skin penetration while maximizing the total amount of emerged bodily fluid.

Similar to single cutting edge lancets, double cutting edge lancets with smaller tip angles generally penetrate the skin tissue more easily with less tearing and produce less pain. It was discovered for double cutting edge lancets that if the tip angles became too small, the penetration depth required to produce the desired amount of emerged bodily fluid increased to a point where the pain levels became unacceptable. Furthermore, it was realized that when the tip angles became too small, the tendency of the lancet to bend during use increased, thereby increasing pain levels.

Conversely, double cutting edge lancets with larger tip angles tend to require more force to penetrate the skin than double cutting edge lancets with smaller tip angles resulting in additional pressure sensing nerves being affected and increasing the overall sensation of pain. It was expected that the pain levels associated with penetration of lancet 770 would become unacceptable for tip angles 782 and 783 nearing ninety degrees (90°). However, it was discovered testing symmetric tip angle lancets (those where tip angles 782 and 783 are equal) that there was very poor penetration of lancet 770 and unacceptably high levels of pain at tip angles 782 and 783 as unexpectedly low as forty degrees (40°)—when the total included tip angle was eighty degrees (80°). This result was especially surprising since lancet 770 produced similar results at a tip angle 180 of sixty degrees (60°).

Acceptable levels of pain and emerged bodily fluid were realized with symmetric tip angles 782 and 783 of at least fifteen degrees (15°) and at most twenty degrees (20°) for certain example embodiments. It is surprising that the total included tip angle for symmetrical double cutting edge lancets (the sum of tip angles 782 and 783 for lancet 770) that produced good results (at least thirty degrees (30°) and at most forty degrees (40°)) is similar to the total included tip angle (angle 180 for lancet 170) that produced good results with single cutting edge lancets.

It was also further discovered that the lancet thickness 774 can vary from 0.05 to 0.3 mm, and in particular embodiments from 0.05 to 0.15 mm. In certain embodiments, a lancet thickness 774 between 0.05 and 0.1 mm may produce favorable results when flat lancet 770 is formed utilizing photoetching. In other embodiments, a lancet thickness 774 between 0.1 and 0.2 mm may produce favorable results when flat lancet 770 is formed utilizing mechanical stamping.

Yet another example embodiment of a flat lancet 970 is depicted in FIGS. 9 and 10. The flat lancet 970 has two opposing, generally parallel surfaces 972 with two opposing side surfaces 973. The two opposing generally parallel surfaces 972 and the two opposing side surfaces 973 are generally planar, although they may be curved or angled. The mean distance between generally parallel surfaces 972 is the lancet thickness 974, and the mean distance between side surfaces 973 is the lancet width 975. Generally, the flat lancet 970 has the lancet thickness 974 less than the lancet width 975.

The flat lancet 970 is bounded on yet another side by the cutting surfaces 976. The intersection of the cutting surfaces 976 and the generally parallel surfaces 972 form the cutting edges 977. There are two cutting surfaces 976 and four cutting edges 977. Of the four cutting edges 977, one defines a first cutting edge axis 978 and a first cutting edge width 979, and another cutting edge 977 defines a second cutting edge axis 980 and a second cutting edge width 981, as depicted in FIG. 9. In certain embodiments, the cutting edges 977 are symmetrically aligned such that a plane containing the two cutting edges 977 associated with the same side of flat lancet 970, for example, the two cutting edges 977 associated with cutting edge width 981, is perpendicular to the surfaces 972, as depicted in FIG. 10. In other embodiments the cutting edges 977 are offset such that a plane containing the two cutting edges 977 associated with the same side of flat lancet 970 is obliquely angled with respect to the surfaces 972. The first cutting edge axis 978 is offset by a first tip angle 982 from the longitudinal axis 986 of lancet 970; the second cutting edge axis 980 is offset by a second tip angle 983 from the longitudinal axis 986 of the lancet 970.

The longitudinal axis 986 generally defines the long portion of the lancet 970; however, other example embodiments have the longitudinal axis 986 parallel, or tangential, to the thrust axis, where the thrust axis defines the movement of the lancet 970 as it penetrates the skin surface 171.

FIG. 9 depicts the tip angles 982 and 983 as being measured from the sides of the lancet 970, where the sides from which the tip angles 982 and 983 are being measured are parallel to the longitudinal axis 986 of the lancet 970. It is understood that other embodiments of the present invention have, by way of nonlimiting example, the sides angled with respect to the longitudinal axis 986.

The tip of lancet 970 may be symmetric or asymmetric, although in some embodiments a symmetric tip may be preferable for maximizing blood flow while minimizing pain. An asymmetric tip may include, but not be limited to, first cutting edge width 979 and the second cutting edge width 981 being different, cutting surfaces 976 having unequal surface areas, or tip angle 982 and tip angle 983 being different.

The flat lancet 970 minimizes the pain associated with lancing while maximizing the amount of emerged bodily fluid. It was discovered that lancet width 975 can vary from 0.1 to 1.5 mm. For the set lancet width 975, the pain associated with a lancet penetrating the skin may be minimized by varying the tip angles 982 and 983, as well as the lancet thickness 974. The tip angles 982 and 983 are less than eighty degrees (80°). It was also discovered that the tip angles 982 and 983 can individually range from ten to sixty degrees (10° to 60°), and more particularly from fifteen to twenty degrees (15° to 20°) to minimize pain associated with skin penetration while maximizing the total amount of emerged bodily fluid.

When testing symmetric angle tip designs, those where tip angles 982 and 983 are equal, it was discovered that high levels of pain occurred when the tip angles 982 and 983 were at least thirty degrees (30°). It was additionally discovered for symmetric angle tip designs that very high levels of pain and poor tip penetration into the skin resulted when the tip angles 982 and 983 were at least forty degrees (40°). It was further discovered for symmetric angle tip designs that the amount of bodily fluid produced was relatively high while the level of pain was relatively low when the tip angles 982 and 983 were approximately fifteen to twenty degrees (15° to 20°).

It was also discovered that the lancet thickness 974 can vary from 0.05 mm to 0.3 mm, and in particular embodiments from 0.05 to 0.15 mm. In certain embodiments, a lancet thickness 974 between 0.05 and 0.1 mm may produce favorable results when flat lancet 970 is formed utilizing photoetching. In other embodiments, a lancet thickness 974 between 0.1 and 0.2 mm may produce favorable results when flat lancet 970 is formed utilizing mechanical stamping.

The two cutting surfaces 976 may be formed by various methods, such as photoetching, mechanical grinding, mechanical stamping, or laser shaping, by way of nonlimiting examples. The cutting surfaces 976 are generally concave although other example embodiments have cutting surfaces that are generally planar, angled, or convex.

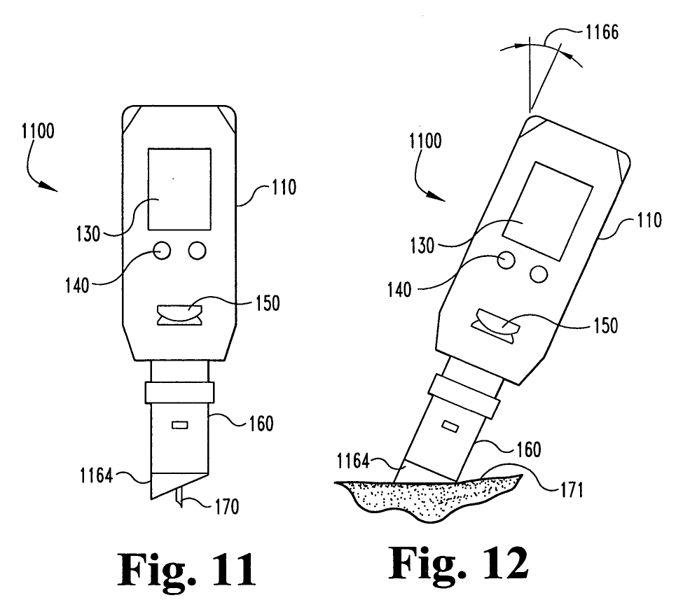

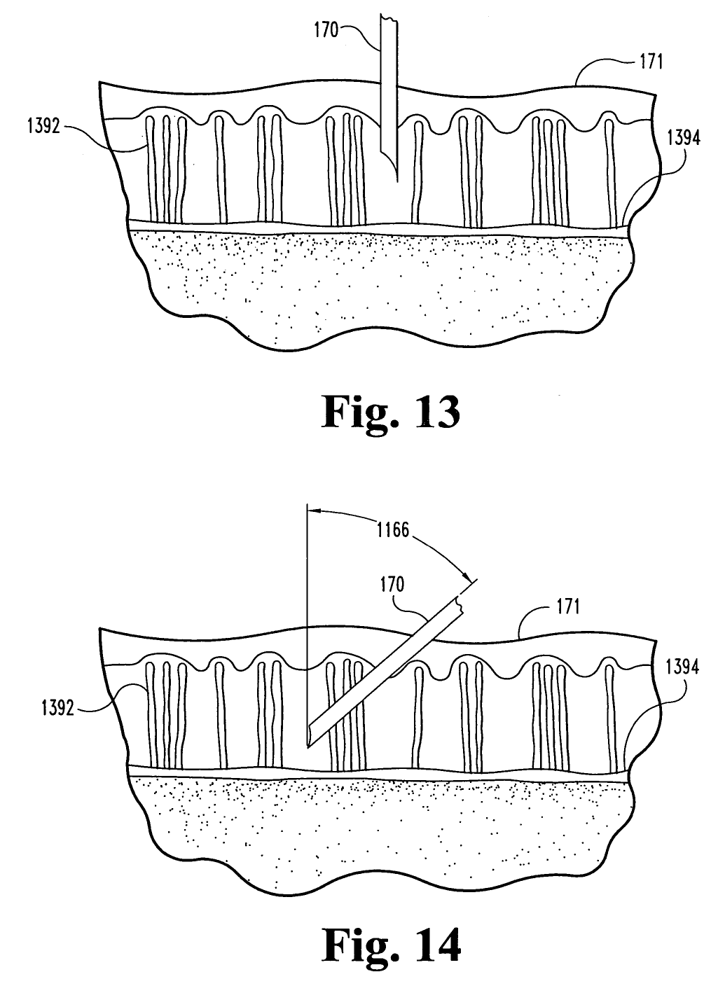

A bodily fluid sampling device 1100 according to another embodiment of the present invention is illustrated in FIGS. 11 and 12. The bodily fluid sampling device 1100 is similar to the bodily fluid sampling device 100 (FIGS. 1 and 2) except as otherwise noted below. The bodily fluid sampling device 1100 utilizes an angled skin contacting portion 1164 where the surface touching the skin surface 171 is oriented at an oblique, or non-orthogonal, angle with respect to the lancet 170.

In use, the sampling device 1100 is positioned on the body such that the angled skin contacting portion 1164 is flat against the skin surface 171, thereby tilting the sampling device 1100 and the lancet 170 with respect to the skin surface 171 by an angle 1166. When actuated, the lancet 170 penetrates the skin surface 171 at the angle 1166. It was discovered that tilting the lancet 170 with respect to the skin surface 171 further minimizes the pain associated with lancing while maximizing the emerged bodily fluid, especially when used in conjunction with different lancet tip designs. The angled skin contacting portion 1164 and the skin contacting portion 162 are interchangeably mountable to the sampling device 1100, although other embodiments permanently attach angled skin contacting portion 1164 to the sampling device 1100. Other embodiments tilt the lancet 170 with respect to the skin surface 171 without tilting the sampling device 1100.

Although not anticipated, it was discovered that penetrating the skin at oblique, or non-orthogonal, angles can increase emerged bodily fluid while not appreciably increasing pain. One potential explanation for this result concerns the structure of tissue as depicted in FIGS. 13 and 14. FIG. 13 depicts the embodiment of the lancet 170 orthogonally penetrating the skin surface 171. Beneath the skin surface 171 are the capillaries 1392 and a superficial vascular plexus 1394. When the lancet 170 orthogonally penetrates the skin surface 171, potential exists for the lancet 170 to rupture none of the capillaries 1392, resulting in little or no emerged blood. One method to ensure blood emerges is for the lancet 170 to penetrate the skin deeper than depicted in FIG. 13 and rupture the superficial vascular plexus 1394; however, this deep penetration method typically results in more pain for the patient. An alternate method to ensure blood emerges is to use a larger lancet to increase the probability of rupturing at least one capillary 1392; however, the larger lancet will also typically result in higher levels of pain.

FIG. 14 depicts the lancet 170 penetrating the skin surface 171 at the oblique lancing angle 1166, that is non-orthogonal relative to the skin surface 171. The oblique lancing angle 1166 can be less than ninety degrees (90°) and greater than zero degrees (0°). It was discovered that the oblique lancing angle 1166, in particular, ranges from forty-five to sixty degrees (45° to 60°) to minimize pain while maximizing the total amount of emerged bodily fluid. Although the lancet 170 is depicted, other lancet designs, such as lancets 570, 770 and 970 by way of nonlimiting example, may also be used. Utilizing this method, the relatively small lancet 170 has a higher probability of rupturing at least one of the capillaries 1392 without requiring deeper penetration or a larger lancet. Due to the higher probability of severing at least one of the capillaries 1392, the oblique penetration of the lancet 170 is advantageous when utilizing alternate sampling sites, such as the palm of the hand, forearm, earlobe and the like.

It was also discovered that the pain level associated with the lancet 170 penetrating the skin is affected by the relationship between the cutting edge axis 178 and the skin surface. In order to maintain the same angular relationship between the cutting edge axis 178 and the skin surface 171, orthogonal and oblique sampling devices may utilize lancets with different tip angles 180 to minimize pain.

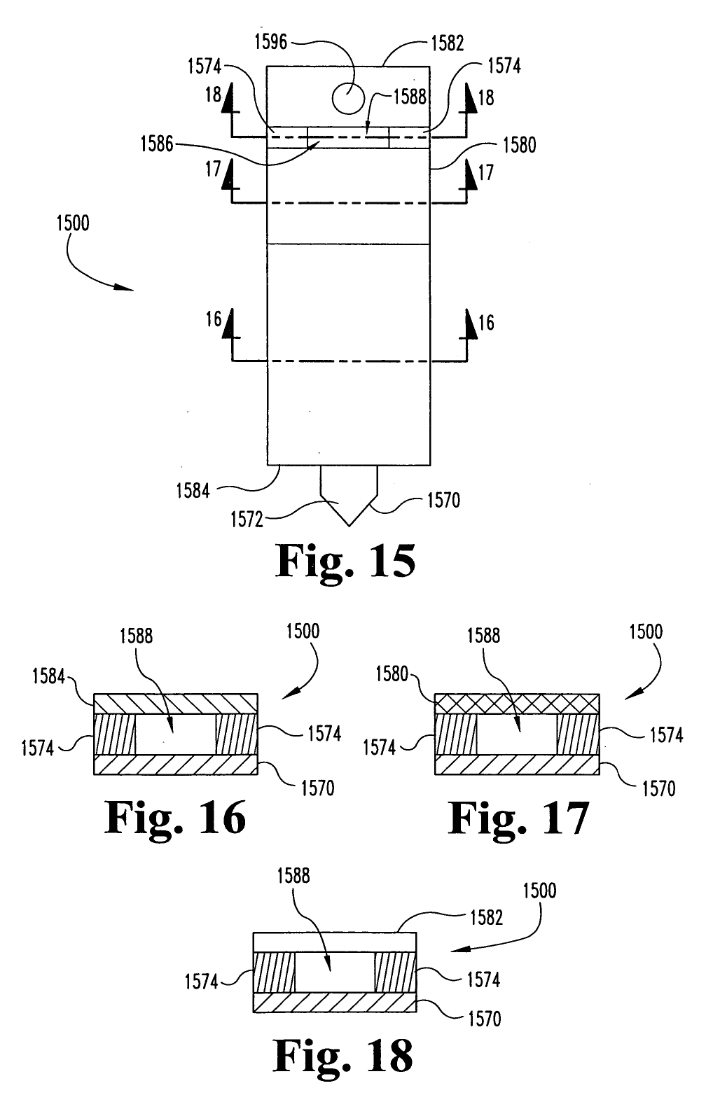

An integrated bodily fluid sampling device 1500 according to yet another embodiment of the present invention is illustrated in FIGS. 15-18. The sampling device 1500 includes a flat lancet 1570 with a cutting portion 1572. The flat lancet 1570 is attached to two spacer layers 1574. The two spacer layers 1574 are attached to a test strip 1580, a first planar member 1582 and a second planar member 1584. The test strip 1580 is located between the first planar member 1582 and the second planar member 1584. A portion of the test strip 1580 abuts against a portion of the second planar member 1584. A gap between the test strip 1580 and the first planar member 1582 forms a vent opening 1586. A capillary channel 1588 is formed by the space between the flat lancet 1570, the two spacer layers 1574, the second planar member 1584 and the test strip 1580. A hole 1596 is included to aid attachment of the integrated bodily fluid sampling device 1500 to another device, such as, by way of nonlimiting example, a device designed to interpret and display the results of the test.

The integrated bodily fluid sampling device 1500 provides a convenient device to lance the body then express, sample and test the emerged bodily fluid, and a device that is easy to manufacture. In use, the integrated bodily fluid sampling device 1500 is moved toward the skin surface until the cutting portion 1572 penetrates the skin surface. As fluid emerges from the rupture formed by the cutting portion 1572, the fluid begins to enter the capillary channel 1588. The capillary channel 1588 draws the bodily fluid up toward, and deposits the bodily fluid on the test strip 1580. Capillary action draws the bodily fluid up the capillary channel 1588. Other embodiments may use different methods of moving the bodily fluid up the capillary channel 1588, such as by way of nonlimiting example, vacuum pressure. The vent opening 1586 allows air to escape from the capillary channel 1588 and minimizes creation of a back pressure that would slow the progression of the bodily fluid up the capillary channel 1588. Hydrophilic and hydrophobic coatings may cover all or portions of the walls of the capillary channel 1588 to guide the bodily fluid to the test strip 1580.

The test strip 1580 is typically comprised of a test media that is capable of determining analyte levels in the bodily fluid sample. As should be appreciated, analyte levels can be determined through the chemical, electrical, electromechanical and/or optical properties of the bodily fluid sample collected on the test media, to name a few. Typically, test strip 1580 is sensitive to thermal and/or chemical processes required for sterilization. The sterilization process can affect the results generated by the test media and, therefore, recalibration of the test media may be required after sterilization. A calibration interface, such as the calibration interface 150 of FIG. 1, can be used for recalibration after sterilization. Alternatively, for example, the lancet 170 can be separately sterilized such that the test media does not have to go through the same sterilization process. After sterilization, the lancet 170 can be installed into the sampling device 100, thereby eliminating the need to recalibrate the test media.

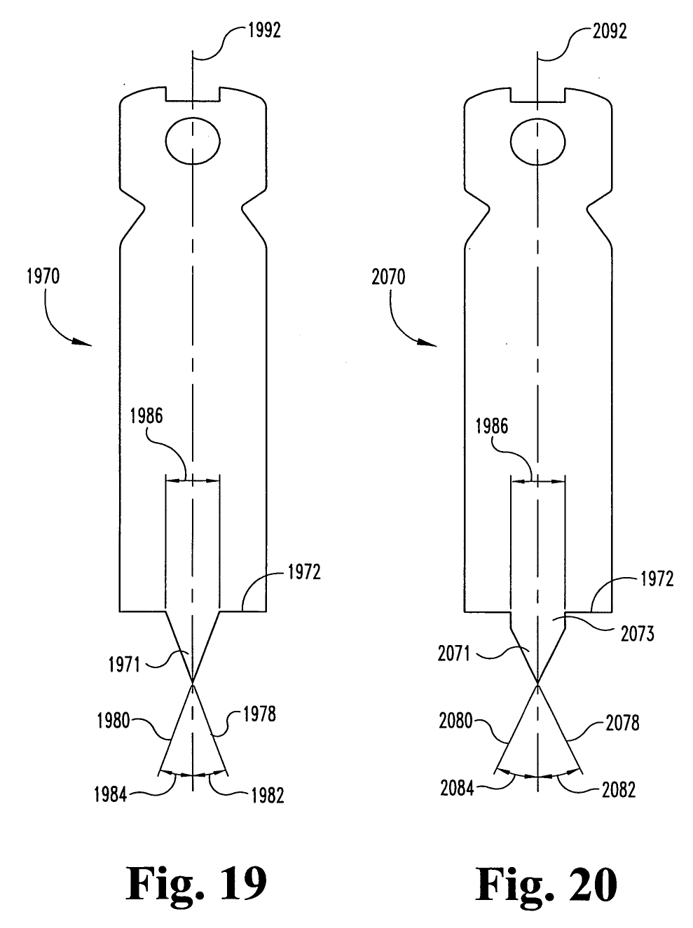

Depicted in FIG. 19 is an example embodiment of a flat lancet 1970. The flat lancet 1970 is generally planar in design and includes a cutting portion 1971 and an abutment portion 1972. The cutting portion 1971 defines a first cutting edge axis 1978 and a second cutting edge axis 1980, which are angularly separated from the longitudinal axis by a first tip angle 1982 and a second tip angle 1984. The cutting portion 1971 further defines a cutting portion width 1986, where the cutting portion width 1986 can vary from 0.1 to 1.5 mm. A longitudinal axis 1992 generally defines the long portion of the lancet 1970; however, other example embodiments have the longitudinal axis 1992 parallel, or tangential, to the thrust axis, where the thrust axis defines the movement of the lancet 1970 as it penetrates the skin surface.

The flat lancet 1970, and in particular the cutting portion 1971, may be formed by various methods, such as photoetching, mechanical grinding, mechanical stamping, or laser shaping, by way of nonlimiting examples. The flat lancet 1970 minimizes the pain associated with lancing while maximizing the amount of emerged bodily fluid. The depicted flat lancet 1970 is a representation of a lancet design where the first tip angle 1982 and the second tip angle 1984 are relatively small, such as by way of nonlimiting example, each being at least ten degrees (10°) and at most fifteen degrees (15°).

The flat lancet 1970 may be used in conjunction with devices such as the integrated sampling device 1500 and the sampling device 100. In use, the flat lancet 1970 moves the cutting portion 1971 to penetrate the skin surface. The abutment portion 1972 abuts against a corresponding abutment portion in the test device to stop the movement of the flat lancet 1970 after the cutting portion 1971 has penetrated the skin a particular distance. The corresponding abutment portion in the test device can be adjustable to allow selectable variations in the penetration depth.

An example embodiment of a flat lancet 2070 is depicted in FIG. 20. The flat lancet 2070 is similar to the flat lancet 1970 except as otherwise indicated. The flat lancet 2070 includes a cutting portion 2071 and a shank portion 2073. Cutting portion 2071 defines a first cutting edge axis 2078 and a second cutting edge axis 2080, which are angularly separated from the longitudinal axis by a first tip angle 2082 and a second tip angle 2084. A longitudinal axis 2092 generally defines the long portion of the lancet 2070; however, other example embodiments have the longitudinal axis 2092 parallel, or tangential, to the thrust axis, where the thrust axis defines the movement of the lancet 2070 as it penetrates the skin surface.

The flat lancet 2070 minimizes the pain associated with lancing while maximizing the amount of emerged bodily fluid. The depicted flat lancet 2070 is a representation of a lancet design where the first tip angle 2082 and the second tip angle 2084 are somewhat larger than the first tip angle 1982 and the second tip angle 1984 of flat lancet 1970. By way of nonlimiting example, the first tip angle 2082 and the second tip angle 2084 are at least fifteen degrees (15°) and at most twenty degrees (20°).

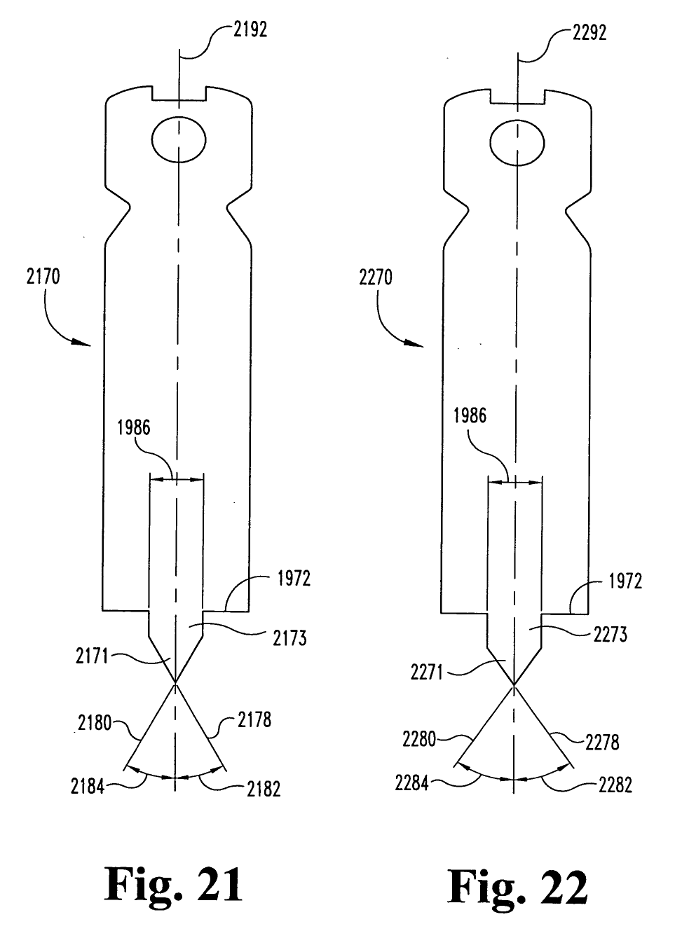

Referring to FIG. 21, an example embodiment of a flat lancet 2170 is depicted. The flat lancet 2170 is similar to the flat lancet 1970 except as otherwise indicated. The flat lancet 2170 includes a cutting portion 2171 and a shank portion 2173. Cutting portion 2171 defines a first cutting edge axis 2178 and a second cutting edge axis 2180, which are angularly separated from the longitudinal axis by a first tip angle 2182 and a second tip angle 2184. A longitudinal axis 2192 generally defines the long portion of the lancet 2170; however, other example embodiments have the longitudinal axis 2192 parallel, or tangential, to the thrust axis, where the thrust axis defines the movement of the lancet 2170 as it penetrates the skin surface.

The flat lancet 2170 minimizes the pain associated with lancing while maximizing the amount of emerged bodily fluid. The depicted flat lancet 2170 is a representation of a lancet design where the first tip angle 2182 and the second tip angle 2184 are somewhat larger than the first tip angle 2082 and the second tip angle 2084 of flat lancet 2070. By way of nonlimiting example, the first tip angle 2182 and the second tip angle 2184 are at least twenty degrees (20°) and at most twenty-five degrees (25°).

Depicted in FIG. 22 is an example embodiment of a flat lancet 2270 is depicted. The flat lancet 2270 is similar to the flat lancet 1970 except as otherwise indicated. The flat lancet 2270 includes a cutting portion 2271 and a shank portion 2273. Cutting portion 2271 defines a first cutting edge axis 2278 and a second cutting edge axis 2280, which are angularly separated from the longitudinal axis by a first tip angle 2282 and a second tip angle 2284. A longitudinal axis 2292 generally defines the long portion of the lancet 2270; however, other example embodiments have the longitudinal axis 2292 parallel, or tangential, to the thrust axis, where the thrust axis defines the movement of the lancet 2270 as it penetrates the skin surface.

The flat lancet 2270 minimizes the pain associated with lancing while maximizing the amount of emerged bodily fluid. The depicted flat lancet 2270 is a representation of a lancet design where the first tip angle 2282 and the second tip angle 2284 are somewhat larger than the first tip angle 2182 and the second tip angle 2184 of flat lancet 2170. By way of nonlimiting example, the first tip angle 2282 and the second tip angle 2284 are at least twenty-five degrees (25°) and at most thirty degrees (30°).

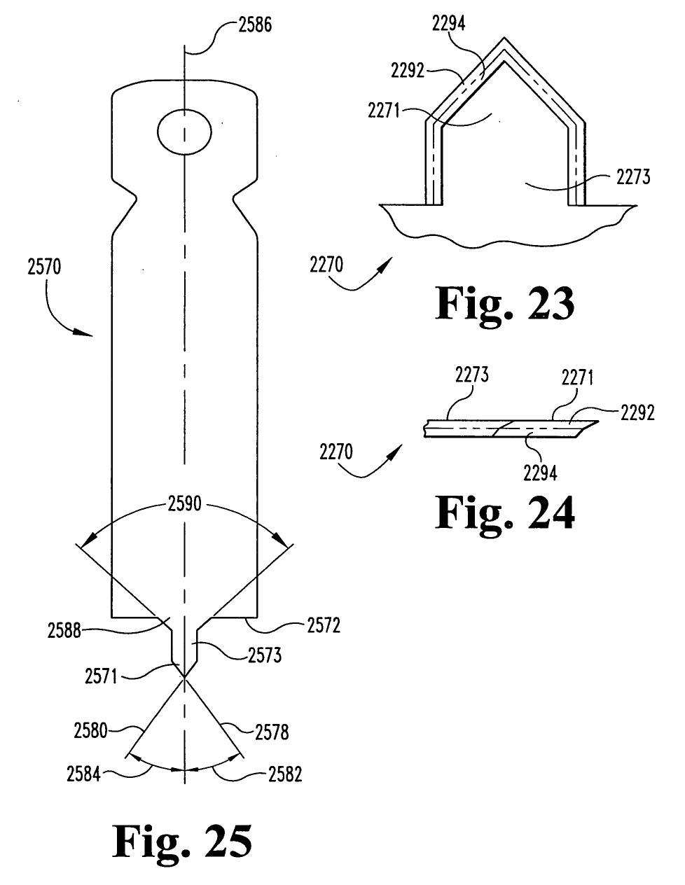

FIGS. 23 and 24 depict the cutting portion 2271 and the shank portion 2273 of the flat lancet 2270. In this example embodiment, the lancet tip comprises at least a first cutting surface 2292 and a second cutting surface 2294, which are generally planar and angled with respect to each other, although other example embodiments have cutting surfaces that are curved.

The cutting surfaces 2292 and 2294 may be formed by various methods, such as photoetching, mechanical grinding, mechanical stamping, or laser shaping, by way of nonlimiting examples. As an example, photoecthing, as described above, may be used to produce the depicted example tip design of flat lancet 2270, which may be called a “boot-shaped” design (FIG. 24). In one embodiment, an offset-type photoetching technique, where the masking on both sides of the lancet are offset, can be utilized to produce the boot-shaped tip. Although the offset-type photoetching has certain advantages, the need for precise offsetting of the photoresist between the two sides can require a greater amount of time to ensure precise placement of the photoresist and can result in a larger percentage of incorrectly formed lancets if the placement of photoresist is not precisely controlled. The angled cutting surfaces 2292 and 2294 are advantageous in further minimizing the pain associated with the lancet 2270 penetrating the skin.

Yet another example embodiment of a flat lancet 2570 is depicted in FIG. 25. The flat lancet 2570 is similar to the flat lancet 1970 except as otherwise indicated. The flat lancet 2570 includes a cutting portion 2571 and a shank portion 2573. Cutting portion 2571 defines a first cutting edge axis 2578 and a second cutting edge axis 2580, which are angularly separated from the longitudinal axis by a first tip angle 2582 and a second tip angle 2584. By way of nonlimiting example, the first tip angle 2582 and the second tip angle 2584 are at least fifteen degrees (15°) and at most twenty degrees (20°). A longitudinal axis 2586 generally defines the long portion of the lancet 2570; however, other example embodiments have the longitudinal axis parallel, or tangential, to the thrust axis, where the thrust axis defines the movement of the lancet 2570 as it penetrates the skin surface. The flat lancet 2570 further includes a shank tapered portion 2588, which defines a shank tapered portion angle 2590. This example embodiment of flat lancet 2570 is sometimes referred to as a “double-shank double-edge” flat lancet. The flat lancet 2570 minimizes the pain associated with lancing while maximizing the amount of emerged bodily fluid.

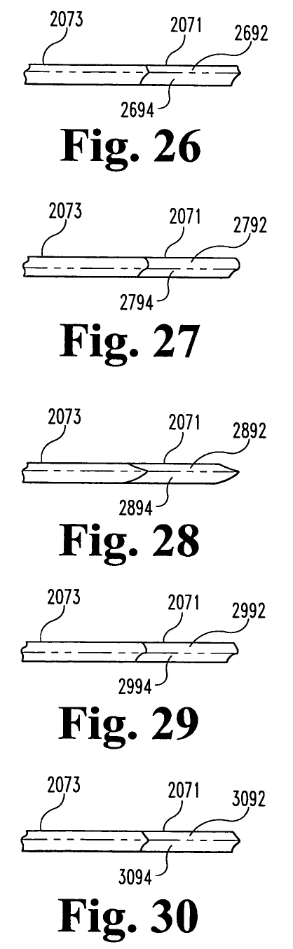

FIG. 26 depicts an example side profile of the cutting portion 2071 and the shank portion 2073 of the flat lancet 2070, although this example side profile is not limited to the example embodiment flat lancet 2070. In this example embodiment, tip angles 2082 and 2084 both equal twenty degrees (20°) and the lancet tip comprises at least a first cutting surface 2692 and a second cutting surface 2694, which are both concavely curved and are inclined with respect to one another. The angled cutting surfaces 2692 and 2694 are advantageous in further minimizing the pain associated with the lancet 2070 penetrating the skin. Other example embodiments have cutting surfaces that are planar or a combination of planar and curved.

The cutting surfaces 2692 and 2694 may be formed by various methods, such as photoetching, mechanical grinding, mechanical stamping, or laser shaping, by way of nonlimiting examples. Furthermore, the side profile of cutting surfaces 2692 and 2694 can generally be controlled using these methods to achieve at least one of an increase in emerged bodily fluid and a decrease pain. By way of nonlimiting example, utilizing a wet (liquid) etchant with photoetching generally results in undercutting. Undercutting occurs since the wet etchant tends to be isotropic in that it removes material at the same rate in all directions and tends to remove material directly under the photoresist. The undercutting causes the cross-sectional amount of removed lancet material to vary as the wet etchant penetrates the lancet material, which can be used to control the side profile of cutting surface 176.

Although not expected, it was discovered that undercutting, which is frequently regarded as an undesirable side effect of wet etching, can produce advantageous results. For example, undercutting can produce enhanced tip sharpness by tapering the cutting surface. Greater tapering generally results in a more pointed cutting edge and a sharper lancet, and is advantageous in further minimizing the pain associated with the lancet 2070 penetrating the skin. However, the amount of tapering must be limited to prevent the cutting edge from becoming so pointed and thin that it bends when penetrating the skin.

Another unexpected result occurred when the amount of time the lancet material was exposed to the etchant was decreased. Generally, it is desirable to allow the etchant to remove all of the lancet material not covered by the photoresist since it generally produces predictably consistent lancet edges. Furthermore, preventing the etchant from completely removing the lancet material not covered by the photoresist requires the lancet to be broken away from the remaining lancet material. However, this breaking away of the lancet produces a fractured surface that can result in favorable tip shapes and side profiles, such as those depicted in FIGS. 26-30. Utilizing this technique, pointed side profiles (for example, FIGS. 8 and 26-30) can be produced using single sided photoetching in addition to the double sided technique frequently used.

Other surprising results were encountered when attempting to control the side profile of cutting surfaces 2692 and 2694. The speed at which the lancet material passes through the etchant, for example when the etchant is applied using a spray, affects the side profile of the cutting surfaces 2692 and 2694. The orientation of the lancet material as the etchant is applied also affects the side profile of the cutting surfaces 2692 and 2694. For example, different side profiles were realized when the lancet material was passed through an etchant spray in orientations that differed by ninety degrees (90°)—one orientation producing a “boot-shaped” side profile (for example, FIG. 24) and the other orientation producing a pointed side profile (for example, FIGS. 8 and 26-30). It was still additionally discovered that the pressure used to spray the etchant affected the side profile of the cutting surfaces 2692 and 2694.

FIG. 27 depicts an example side profile of the cutting portion 2071 and the shank portion 2073 of the flat lancet 2070, which is similar to the side profile depicted in FIG. 26 except as otherwise illustrated and discussed. In this example embodiment, tip angles 2082 and 2084 both equal twenty degrees (20°) and the lancet tip comprises at least a first convexly curved cutting surface 2792 and a second concavely curved cutting surface 2794, which are inclined with respect to one another. The angled cutting surfaces 2792 and 2794 are advantageous in further minimizing the pain associated with the lancet 2070 penetrating the skin. Other example embodiments have cutting surfaces that are planar or a combination of planar and curved.

The cutting surfaces 2792 and 2794 may be formed by various methods, such as photoetching, mechanical grinding, mechanical stamping, or laser shaping, by way of nonlimiting examples. When utilizing photoetching, either single or double sided photoetching may be used and various production parameters may be adjusted to produce the depicted side profile. Example production parameters are the speed and orientation of the lancet material as it passes through the etchant, the pressure used to spray the etchant, and the length of time the etchant is allowed to dissolve the lancet material.

FIG. 28 depicts an example side profile of the cutting portion 2071 and the shank portion 2073 of the flat lancet 2070, which is similar to the side profile depicted in FIG. 26 except as otherwise illustrated and discussed. In this example embodiment, tip angles 2082 and 2084 both equal fifteen degrees (15°) and the lancet tip comprises at least a first concavely curved cutting surface 2892 and a second convexly curved cutting surface 2894, which are inclined with respect to one another. The angled cutting surfaces 2892 and 2894 are advantageous in further minimizing the pain associated with the lancet 2070 penetrating the skin. Other example embodiments have cutting surfaces that are planar or a combination of planar and curved.

The cutting surfaces 2892 and 2894 may be formed by various methods, such as photoetching, mechanical grinding, mechanical stamping, or laser shaping, by way of nonlimiting examples. When utilizing photoetching, either single or double sided photoetching may be used and various production parameters may be adjusted to produce the depicted side profile. Example production parameters are the speed and orientation of the lancet material as it passes through the etchant, the pressure used to spray the etchant, and the length of time the etchant is allowed to dissolve the lancet material.

FIG. 29 depicts an example side profile of the cutting portion 2071 and the shank portion 2073 of the flat lancet 2070, which is similar to the side profile depicted in FIG. 26 except as otherwise illustrated and discussed. In this example embodiment, tip angles 2082 and 2084 both equal fifteen degrees (15°) and the lancet tip comprises at least a first convexly curved cutting surface 2992 and a second concavely curved cutting surface 2994, which are inclined with respect to one another. The angled cutting surfaces 2992 and 2994 are advantageous in further minimizing the pain associated with the lancet 2070 penetrating the skin. Other example embodiments have cutting surfaces that are planar or a combination of planar and curved.

The cutting surfaces 2992 and 2994 may be formed by various methods, such as photoetching, mechanical grinding, mechanical stamping, or laser shaping, by way of nonlimiting examples. When utilizing photoetching, either single or double sided photoetching may be used and various production parameters may be adjusted to produce the depicted side profile. Example production parameters are the speed and orientation of the lancet material as it passes through the etchant, the pressure used to spray the etchant, and the length of time the etchant is allowed to dissolve the lancet material.

FIG. 30 depicts an example side profile of the cutting portion 2071 and the shank portion 2073 of the flat lancet 2070, which is similar to the side profile depicted in FIG. 26 except as otherwise illustrated and discussed. In this example embodiment, tip angles 2082 and 2084 both equal seventeen and one half degrees (17.5°) and the lancet tip comprises at least a first approximately planar cutting surface 3092 and a second concavely curved cutting surface 3094, which are inclined with respect to one another. The angled cutting surfaces 3092 and 3094 are advantageous in further minimizing the pain associated with the lancet 2070 penetrating the skin. Other example embodiments have cutting surfaces that are curved, planar or a combination of planar and curved.

The cutting surfaces 3092 and 3094 may be formed by various methods, such as photoetching, mechanical grinding, mechanical stamping, or laser shaping, by way of nonlimiting examples. When utilizing photoetching, either single or double sided photoetching may be used and various production parameters may be adjusted to produce the depicted side profile. Example production parameters are the speed and orientation of the lancet material as it passes through the etchant, the pressure used to spray the etchant, and the length of time the etchant is allowed to dissolve the lancet material.

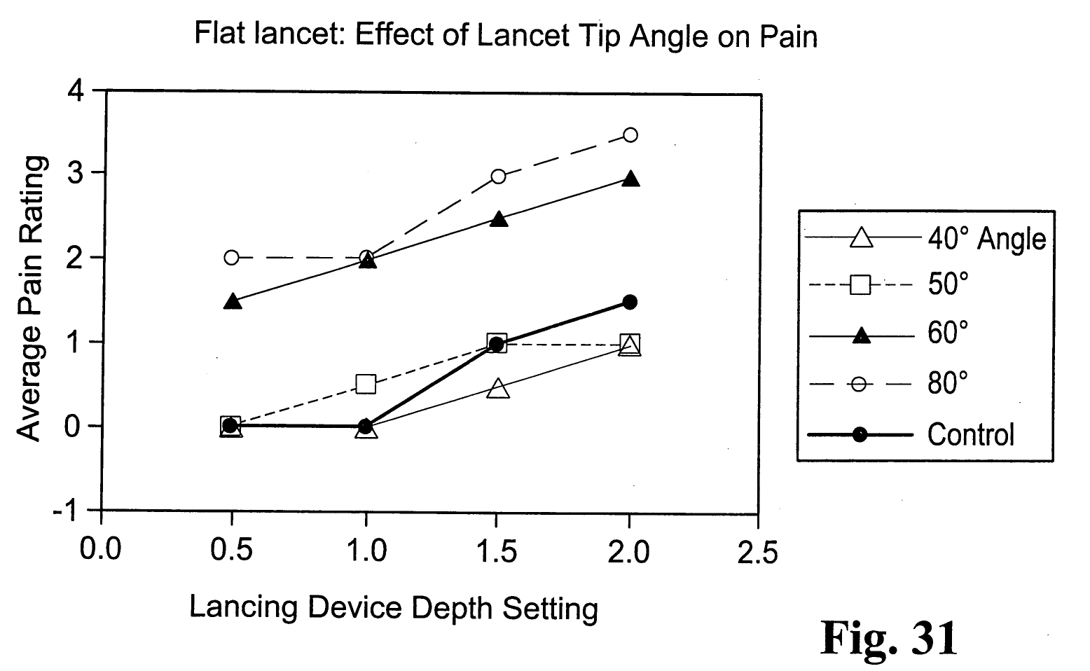

FIG. 31 is a graphic representation of example test data depicting the variation of blood volume with lancet tip angle. The lancets used to obtain the example test data are similar in design to the lancets depicted in FIGS. 19-22. The plot depicts data for four separate tip angles (forty, fifty, sixty and eighty degrees (40°, 50°, 60° and 80°)) and a control round lancet. The plot reflects that the pain increases as the tip angle increases and as the depth setting increases. The plot also reflects the surprising result that the pain ratings for the forty degree (40°) and fifty degree (50°) tip angle lancets do not increase as rapidly as the pain rating for the control round lancet and that the level of pain for the forty degree (40°) and fifty degree (50°) tip angle lancets is actually less that that for the control round lancet at higher depth settings.

While the invention has been illustrated and described in detail in the drawings and foregoing description, the same is to be considered as illustrative and not restrictive in character, it being understood that only example embodiments have been shown and described and that all changes and modifications that come within the spirit of the invention are desired to be protected. All publications, patents, and patent applications cited in this specification are herein incorporated by reference as if each individual publication, patent, or patent application were specifically and individually indicated to be incorporated by reference and set forth in its entirety herein.

What is claimed is:

1. An apparatus, comprising:
a flat lancet adapted to form a breach in the skin surface, said lancet including a longitudinal axis, a thickness being at least 0.05 millimeters and at most 0.15 millimeters to minimize the pain associated with the lancet penetrating the skin, a width being at least 0.1 millimeters and at most 1.5 millimeters, a first cutting edge adapted to penetrate the skin, and a second cutting edge adapted to penetrate the skin, wherein said first and second cutting edges are each inclined at least thirty degrees (30°) and at most forty degrees (40°) from said longitudinal axis; and
an actuation member attached to said flat lancet, wherein said actuation member moves said first cutting edge to penetrate the skin.

2. An apparatus, comprising:
a flat lancet adapted to form a breach in the skin surface, said lancet including a longitudinal axis, a thickness, and a first cutting edge adapted to penetrate the skin, wherein said first cutting edge is inclined less than eighty degrees (80°) from said longitudinal axis and said thickness is at least 0.05 millimeters and at most 0.3 millimeters to minimize the pain associated with the lancet penetrating the skin; and
an actuation member attached to said flat lancet, wherein said actuation member moves said first cutting edge to penetrate the skin.

3. The apparatus of claim 2, wherein said first cutting edge is inclined at least ten degrees (10°) and at most sixty degrees (60°) from said longitudinal axis.

4. The apparatus of claim 3, wherein said first cutting edge is inclined at least thirty degrees (30°) and at most forty degrees (40°) from said longitudinal axis.

5. The apparatus of claim 2, wherein said thickness is at least 0.05 millimeters and at most 0.15 millimeters.

6. The apparatus of claim 2, wherein said longitudinal axis is oriented less than ninety degrees (90°) from the skin surface when said flat lancet penetrates the skin.

7. The apparatus of claim 6, wherein said longitudinal axis is oriented at least forty-five degrees (45°) and at most sixty degrees (60°) from the skin surface when said flat lancet penetrates the skin.

8. The apparatus of claim 2, wherein said flat lancet further comprises a shank.

9. The apparatus of claim 2 further comprising a depth stop, wherein said depth stop prevents said cutting portion from penetrating the skin past a predetermined depth.

10. The apparatus of claim 2, further comprising an expressing device, wherein said expressing device urges bodily fluid from the breach in the skin surface.

11. The apparatus of claim 10, further comprising a test device, wherein said test device tests the bodily fluid for particular properties.

12. The apparatus of claim 11, further comprising a sampling device, wherein said sampling device moves the bodily fluid from the breach in the skin surface to said test device.

13. The apparatus of claim 2, wherein said first cutting edge is concave.

14. The apparatus of claim 2, wherein said flat lancet further includes a width, wherein said width is at least 0.1 millimeters and at most 1.5 millimeters.

15. The apparatus of claim 2, further comprising a second cutting edge adapted to penetrate the skin, wherein said second cutting edge is inclined less than eighty degrees (80°) from said longitudinal axis.

16. The apparatus of claim 15, wherein said second cutting edge is inclined at least ten degrees (10°) and at most sixty degrees (60°) from said longitudinal axis.

17. The apparatus of claim 16, wherein said second cutting edge is inclined at least fifteen degrees (15°) and at most twenty degrees (20°) from said longitudinal axis.

18. The apparatus of claim 15, wherein said first and second cutting edges are symmetrically inclined from said longitudinal axis.

19. The apparatus of claim 15, wherein said second cutting edge is concave.

20. The apparatus of claim 15 further comprising a cutting surface, wherein said second cutting edge is similarly shaped to said first cutting edge and said cutting surface is bounded on two sides by said first cutting edge and said second cutting edge.

21. The apparatus of claim 20, wherein said cutting surface is concave.

22. The apparatus of claim 15 further comprising a third cutting edge, a fourth cutting edge, a first cutting surface, and a second cutting surface; wherein said first cutting surface is bounded on two sides by said first cutting edge and said third cutting edge, and said second cutting surface is bounded on two sides by said second cutting edge and said fourth cutting edge.

23. The apparatus of claim 22, wherein said third cutting edge is similarly shaped to said first cutting edge and said fourth cutting edge is similarly shaped to said second cutting edge.

24. The apparatus of claim 23, wherein said second cutting surface is concave.

25. A method, comprising:
placing a sampling device in contact with the skin, the sampling device including a generally planar lancet, the generally planar lancet having a thickness of at least 0.05 millimeters and at most 0.3 millimeters, and the generally planar lancet having at least one cutting edge;
moving the generally planar lancet in relation to the skin surface and along a translation axis; and
penetrating the skin surface at a penetration site with the generally planar lancet wherein the at least one cutting edge is inclined less than eighty degrees (80°) from the translation axis.

26. The method of claim 25, wherein the at least one cutting edge is inclined at least ten degrees (10°) and at most sixty degrees (60°) from the translation axis.

27. The method of claim 26, wherein the at least one cutting edge is inclined at least thirty degrees (30°) and at most forty degrees (40°) from the translation axis.

28. The method of claim 25, wherein said penetrating occurs with the translation axis oriented less than ninety degrees (90°) from the skin surface.

29. The method of claim 28, wherein said penetrating occurs with the translation axis oriented at least forty-five degrees (45°) and at most sixty degrees (60°) with respect to the skin surface at the point where the translation axis intersects the skin surface.

30. The method of claim 28, wherein the translation axis is curved.

31. The method of claim 25, wherein the generally planar lancet thickness is at least 0.05 millimeters and at most 0.15 millimeters.

32. The method of claim 25, wherein said penetrating terminates at a predetermined depth.

33. The method of claim 25, further comprising urging bodily fluid from the penetration site.

34. The method of claim 33, further comprising transporting the bodily fluid to a test device.

35. The method of claim 34, further comprising testing the fluid released after said penetrating.

36. The method of claim 25, further comprising removing the generally planar lancet from the skin surface.

37. An apparatus for extracting bodily fluid, comprising:
an incision forming member adapted for cutting the skin surface, wherein said incision forming member includes a side surface and at least one cutting facet inclined less than eighty degrees (80°) from said side surface, and wherein said incision forming member has a thickness greater than 0.05 millimeters and less than 0.3 millimeters; and
means for moving at least a portion of said incision forming member below the skin surface at an incision location.

38. The apparatus of claim 37, wherein said at least one cutting facet is inclined at least thirty degrees (30°) and at most forty degrees (40°) from said side surface.

39. The apparatus of claim 37, wherein said incision forming member is at least 0.05 millimeters and at most 0.15 millimeters thick.

40. The apparatus of claim 37, wherein said means for moving includes penetrating the skin surface at an angle less than ninety degrees (90°).

41. The apparatus of claim 37 further comprising an expressing member, wherein said expressing member expresses bodily fluid from the incision location.

42. The apparatus of claim 41 further comprising a test device, wherein said test device tests the bodily fluid for particular properties.

43. The apparatus of claim 42 further comprising means for transporting the bodily fluid to said test device.

44. The apparatus of claim 37, wherein said means for moving includes removing at least a portion of said incision forming member from below the skin surface.

45. The apparatus of claim 37, wherein said means for moving includes a means for moving said incision forming member along a curved path.

46. An apparatus for rupturing the skin, comprising:
a flat lancet for rupturing the skin at a rupture site, said flat lancet comprising a first side and a cutting surface, said cutting surface produced by photoetching, wherein said photoetching is performed on said first side; and
an actuation member, wherein said actuation member moves said flat lancet to rupture the skin at the rupture site.

47. The apparatus of claim 46, wherein said cutting surface is concave.

48. The apparatus of claim 46 further comprising a second side, wherein said photoetching is further performed on said second side.

49. The apparatus of claim 48 further comprising a first cutting edge and a second cutting edge, wherein said first cutting edge is formed by the intersection of said cutting surface and said first side, and said second cutting edge is formed by the intersection of said cutting surface and said second side, and wherein the plane intersecting said first cutting edge and said second cutting edge is perpendicular to said first side.

50. The apparatus of claim 46, wherein said actuation member moves said flat lancet along a curved path.

51. The apparatus of claim 46, wherein said actuation member moves said flat lancet to rupture the skin at an angle less than ninety degrees (90°) from the skin surface.

52. The apparatus of claim 51, wherein said actuation member moves said flat lancet to rupture the skin at an angle at least forty-five degrees (45°) and at most sixty degrees (60°) from the skin surface.

53. The apparatus of claim 46, wherein said actuation member removes said flat lancet from the rupture site.

54. The apparatus of claim 46 further comprising an urging member for urging bodily fluid from the rupture site.

55. The apparatus of claim 54 further comprising a test device for testing the bodily fluid for particular properties.

56. The apparatus of claim 55 further comprising a sampling member for transporting the bodily fluid from the rupture site to the test device.
